(19) United States
(12) Patent Application Publication
(10) Pub. No.: US 2015/0323517 A1
(43) Pub. Date: Nov. 12, 2015

(54) Method And System For Spatially Resolved Wettability Determination

(71) Applicant: Ingrain, Inc., Houston, TX (US)

(72) Inventor: Kathryn Elizabeth Washburn, Houston, TX (US)

(73) Assignee: INGRAIN, INC., Houston, TX (US)

(21) Appl. No.: 14/704,996

(22) Filed: May 6, 2015

Related U.S. Application Data
(60) Provisional application No. 61/989,618, filed on May 7, 2014.

(51) Int. Cl. G01N 33/24

(52) U.S. Cl. CPC G01N 33/246

(57) ABSTRACT

A method which allows for determining wettability with spatial resolution of porous materials or other materials is provided. The method can provide an absolute method of quantifying wettability, and which is a spatially resolved method. A system for performing the method also is provided.

This application claims the benefit under 35 U.S.C. §119(e) of prior U.S. Provisional Patent Application No. 61/989,618, filed May 7, 2014, which is incorporated in its entirety by reference herein.

### FIELD OF THE INVENTION

The present invention relates to spatially resolved wettability determination and, more particularly, to a method for determining wettability with spatial resolution, and a system for making such determinations, which can be used for determining wettability of porous materials, such as porous geological materials, or other materials.

### BACKGROUND OF THE INVENTION

Surface wettability is an important property that influences hydrocarbon flow and production. Wettability is a very important factor in determining the amount of hydrocarbon that may exist in a reservoir, the rate and ease of hydrocarbon production and the ultimate recovery level of hydrocarbons from the reservoir. However, wettability is still poorly understood within porous materials.

Wettability is a surface's preference to be in contact with one fluid over another. Wettability may arise from the surface composition, deposits on the surface and the surface structure. The simplest test for wettability is the contact angle test, where two fluids are placed in contact with the surface and then the contact angle between the surface and a fluid is measured. If the contact angle is low (θ<75°), then the fluid is considered to be wetting. If the contact angle is high (θ>105°), then the fluid is considered non-wetting. If the contact angle is approximately 90° (75°<θ<105°), then the fluid is considered to be neutral wet; neither fluid has a strong preference to be in contact with the surface.

Despite its importance, no good way of measuring wettability within porous materials currently exists. Current methods of measuring wettability for geological samples tend to be unreliable, do not give an absolute wettability value, only relative, and only give a bulk wettability value for the whole sample despite that wettability may vary throughout the pore space.

Wettability testing within porous media is significantly more difficult for numerous reasons. Firstly, direct observation of the fluid contact angle is not possible in many systems due to sample opaqueness and size. Secondly, surface roughness makes it difficult to determine what the true contact angle is. Lastly, the wettability of the sample may not be constant and may vary throughout the sample depending on mineral composition or between pores of similar mineral composition but differing sizes.

The two standard methods within the oil industry of determining the wettability within a porous material are the Amott-Harvey Test and the United States Bureau of Mines (USBM) test. The Amott-Harvey test measures wettability by taking a rock core at irreducible water saturation and placing it in water. The amount of water that is spontaneously imbibed is measured. Once spontaneous imbibition has ended, the sample is placed into a centrifuge or flooding apparatus and the amount of water that can be forcibly imbibed into the core is measured. The process is then repeated for oil; the amount of oil that will spontaneously imbibe in the rock is measured and then the amount of oil that can be forcibly imbibed into the core is measured.

The Amott-Harvey test gives the water wetting index by calculating the ratio of the amount of water spontaneously imbibed versus the total amount of water imbibed. Similarly, it gives an oil wetting index by the ratio of the spontaneously imbibed oil to the total amount of oil imbibed. Samples that imbibe neither fluid are considered to be neutral wet. The USBM method for calculation of wettability index does not include the spontaneous imbibition and simply measures the log of the areas between the two forced imbibition steps. Despite their similarities, the two methods may show significant divergence in results for neutral wet samples.

The Amott-Harvey and USBM methods are frequently combined due to their significant similarities. Neither method gives an absolute value of wettability, but are relative measures that allow petrophysicsts to compare the wettability behaviour between different plugs.

Other methods have been developed to try to estimate wettability, however none of these have been considered reliable enough for widespread use. Nuclear magnetic resonance (NMR) is one of the more commonly used alternative techniques. The relaxation rate of the NMR signal depends on contact of fluid with the surfaces. Shifts in the relaxation times of different types of fluids or measurement of the amount of internal gradients experienced by different fluids can be used to estimate wettability. However, these methods are still relative.

### SUMMARY OF THE INVENTION

A feature of the present invention is a method for determining wettability with spatial resolution of porous materials or other materials.

A further feature of the present invention is a system for making such determinations.

Another feature of the present invention is to provide such methods and systems to provide reliable determinations of wettability for porous geological samples, and which give absolute wettability values for the samples.

To achieve these and other advantages and in accordance with the purposes of the present invention, as embodied and broadly described herein, the present invention relates, in part, to a method for determining surface wettability of at least one sample, comprising a) obtaining spectral data on the at least one sample, b) obtaining spatial information on at least one sample, c) obtaining wettability information on the at least one sample using the spectral data, and d) determining spatially resolved wettability information for the at least one sample using the wettability information and the spatial information. Spectral and spatial measurements may be performed on the exact same sample or the spectral measurement can be performed on one sample(s) and the spatial measurement performed on a second sample(s) where samples are of similar composition and structure.

A system for performing the method is also provided.

It is to be understood that both the foregoing general description and the following detailed description are exemplary and explanatory only and are intended to provide a further explanation of the present invention, as claimed.

The accompanying figures, which are incorporated in and constitute a part of this application, illustrate various features of the present invention and, together with the description, serve to explain the principles of the present invention. The features depicted in the figures are not necessarily drawn to scale. Similarly numbered elements in different figures represent similar components unless indicated otherwise.

### BRIEF DESCRIPTION OF THE DRAWINGS

FIG. 1 shows a process flow chart of the determining of spatially resolved wettability of a sample according to an example of the present application.

### DETAILED DESCRIPTION OF THE INVENTION

The present invention relates in part to a method which allows for determining wettability with spatial resolution of porous materials or other materials. The method can allow for production of spatially resolved maps of chemical components on the pore surface and provide other advantages and benefits. The method of this invention can help provide absolute values of wettability instead of relative values, and from there, 3D models can be populated with the values obtained. This invention can provide an absolute method of quantifying wettability, and which is a spatially resolved method. The method of the present invention can provide a rapid alternative to previous wettability determination methods which required a long time to perform, and this invention can be beneficial as a stand-alone service as well as improving fluid flow simulations.

The materials, also referred to herein as the samples, to which the present invention can be applied are not necessarily limited. The materials can be porous materials, such as porous geological materials, e.g., rocks. The kinds of rock to which a method of the present invention can be applied are not necessarily limited. The rock sample can be, for example, organic mud rock, shale, carbonate, sandstone, limestone, dolostone, or other porous rocks, or any combinations thereof, or other kinds. Any source of a rock formation sample of manageable physical size and shape may be used with the present invention. Micro-cores, crushed or broken core pieces, drill cuttings, sidewall cores, outcrop quarrying, whole intact rocks, and the like, may provide suitable rock piece or fragment samples for analysis using methods according to the invention.

The present invention relates in part to a method for determining surface wettability of a sample that includes steps of obtaining spectral data on a sample, obtaining spatial information on the sample, obtaining wettability information on the sample using the spectral data, and determining spatially resolved wettability information for the sample using the wettability information and spatial information. Spectral and spatial measurements may be performed on the exact same sample or the spectral measurement can be performed on one sample(s) and the spatial measurement performed on a second sample(s) where samples are of similar composition and structure.

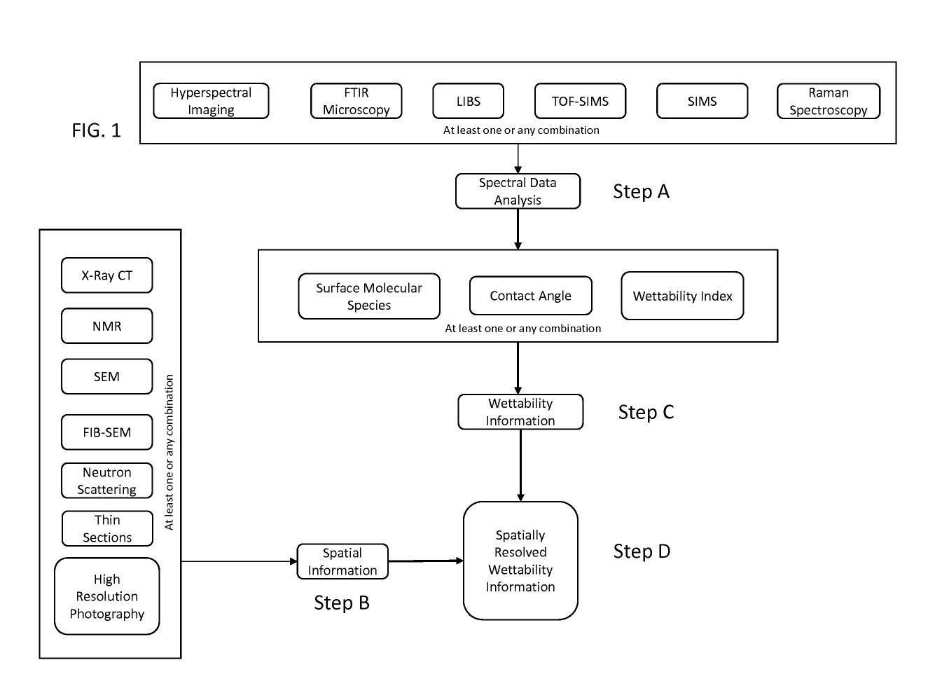

Referring to FIG. 1, a process flow of a method of the present invention is illustrated which includes Steps A, B, C, and D.

In Step A, spectral data is obtained. The spectra are generated by, but not limited to, LIBS, TOF-SIMS, SIMS, FTIR, Raman spectroscopy, Hyperspectral Imaging, or any equipment capable of generating spectral data. More than one spectral data from various methods can be used for analysis.

In Step B, spatial imaging information/data is obtained. Spatial information can be generated by, but not limited to, X-Ray CT scanning, Scanning Electron Microscopy (SEM), Focused Ion Beam-Scanning Electron Microscopy (FIB-SEM), Nuclear Magnetic Resonance (NMR), Neutron Scattering, Thin Sections, High Resolution photography, or any equipment capable of generating spatial information. More than one spatial information from various equipment can be used for analysis.

The samples can undergo spectral measurement and spatial imaging in the same setup, or the samples can undergo spectral measurement and then are transferred to a second setup for spatial imaging, or the samples can undergo spatial imaging and are then transferred to a second equipment for spectral measurement, or the samples can undergo spectral measurement and spatial imaging and one or more intermediate measurements between the two types of measurements. Spectral and spatial measurements may be performed on the exact same sample or the spectral measurement can be performed on one sample(s) and the spatial measurement performed on a second sample(s) where samples are of similar composition and structure.

In Step C, wettability information is compiled from information on contact angle, surface molecular species, wettability index or indices, or any combinations. Any single or combination of Surface Molecular, Contact Angle, or Wettability can be used.

The contact angle can be estimated from the spectral measurements, wherein the contact angle is estimated from molecular species identified from the spectral measurements, or wherein univariate or multivariate analysis can be used to correlate the spectral measurements to contact angle.

As to surface molecular species, the molecular species on the surface that can be identified from spectral measurements are used to correlate the spectral measurements to wettability information derived from Amott-Harvey testing, USBM testing, Amott-USBM testing, NMR measurement, or other wettability description metrics, or wherein univariate or multivariate analysis can be used to correlate the spectral measurements to molecular species.

As to wettability indices, univariate or multivariate analysis is used to correlate the spectral measurements to wettability derived from Amott-Harvey testing, USBM testing, Amott-USBM testing, or NMR measurement, or other wettability description metrics.

In Step D, appropriate spatial distribution of wettability indices in the 2D or 3D models can be determined through image segmentation, assigned manually, determined by capillary pressure simulation or measurements, or determined from previously spatially resolved spectral measurements. Appropriate spatial distribution of surface molecular species in the 2D or 3D models can be determined through image segmentation, assigned manually, by capillary pressure simulation or measurements, or determined from previously spatially resolved spectral measurements. Appropriate spatial distribution of contact angles in the 2D or 3D models can be determined through image segmentation, assigned manually, by capillary pressure simulation or measurements, or determined from previously spatially resolved spectral measurements.

FIG. 1 shows modes of spectral data acquisition which can have the following features and/or others. Time of Flight-Secondary Ion Mass Spectroscopy (TOF-SIMS) uses ions to dislodge molecules from sample surfaces. A variety of ions can be used, including, but not limited to, Ga, Au, Au2, Au3 and C60. Unlike dynamic SIMS, lower energies are used such that molecular structure of the ablated material remains intact. In dynamic SIM, higher energy is used such that the molecular structure is broken and only elements are measured.

For TOF-SIMS, the ablated components are then accelerated to a constant kinetic energy. If kinetic energy is held constant, then the time the species take to travel will vary depending on their mass. By measuring the time of flight, the time it takes for the molecular species to travel though the detector, their mass can be determined. From component mass, the molecular species can then be identified. The measurements are performed as a raster, such that a high resolution map of surface composition can be created. Results have then been analysed using multivariate analysis techniques, such as principle component analysis and partial least squares regression to relate surface composition.

TOF-SIMS has been used to determine contact angle for a variety of different industries such as the semi-conductor and medical industry. The mining industry has used TOF-SIMS to determine surface wettability of geology samples to estimate how well different components will separate during floatation separation.

Dynamic Secondary Mass Spectroscopy uses ions to dislodge molecules from sample surfaces. A variety of ions can be used, including, but not limited to, Ar, Xe, O, SF5 and C60. A mass spectrometer is then used to measure the mass of the produced species. The energy of the ions used is such that the molecular bonds of the surface materials are broken and only the elements are measured. The measurements are performed as a raster, such that a high resolution map of surface composition can be created. Results have then been analysed using multivariate analysis techniques, such as principle component analysis and partial least squares regression to relate surface composition.

Laser induced breakdown spectroscopy (LIBS) uses a laser to ablate a tiny portion of sample. The standard for LIBS uses a q-switched solid state laser that produces a rapid pulse, typically on the order of pico- to nanoseconds in duration. Optics are used to focus the energy onto a single spot on the sample. The laser ablates a small amount of sample at this spot, turning it into a high temperature plasma. The excited atoms then return to a ground state, giving off light of characteristic frequencies. The spot size vaporized by the laser can range in size from a few microns up to hundreds of microns, allowing a large range of resolution and is dependent on the optics of the system. The signal quality improves with larger spot size, but sacrifices resolution. While a small amount of sample is consumed, the amount is so small that it is considered to be negligible and the technique is considered non-destructive. The wavelength of light from the plasma can be in the 200 to 980 nm region. The resulting spectra can be analysed by multivariate data to correlate the spectra to concentration of elements. LIBS has been used previously as a method for mineralogy identification, making it an alternative to X-ray Diffraction (XRD) and X-ray Fluorescence (XRF) methods for mineralogical analysis of samples. It has an advantage over XRF for mineralogical identification because it can measure all elements, whereas XRF is unable to detect light elements.

LIBS is able to perform depth profiling, firing the laser in the same spot and observing the different products that are produced with increased depth. LIBS is also very rapid, only taking per seconds per measurement making it amenable for high-throughput industrial use. LIBS measurements can be rastered to produce a two dimensional map of surface composition.

Fourier transform infrared spectroscopy (FTIR) microscopy combines FTIR measurements with spatial resolution to produce a FTIR spectrum. FTIR works by shining infrared light upon a sample. Depending on the composition of the sample, some wavelengths of light will be absorbed while others will pass through the sample. The transmitted light is then measured to produce a spectra showing an absorption profile as a function of wavelength. Organic matter and inorganic minerals have characteristic absorption profiles which can be used to identify sample constituents. This may be done qualitatively or quantitatively by manual assignment, use of mineral libraries or multivariate analysis. The FTIR microscope advances normal FTIR measurements by combining the technique with an optical microscope such that individual areas of a sample can be selected and FTIR spectra taken, allowing composition at a higher resolution to be determined. Unlike standard FTIR measurements which are normally performed on powders, the FTIR microscopy can be performed on intact samples. Standard procedure for geological FTIR microscopy uses a sample that is polished to produce an even surface. FTIR microscopy can be performed via transmission FTIR, diffuse reflectance infrared fourier transform spectroscopy (DRIFTS), or attenuated total reflectance (ATR) FTIR.

Raman spectroscopy uses monochromatic light, usually from a laser, to excite rotational and vibrational modes in a sample. Raman spectroscopy measures the Raman scattering, the inelastic scattering that occurs when light interacts with matter. When photons from the laser interact with the molecular vibrations in the sample, they change the excitation state of the molecule. As the molecule returns to equilibrium, this results in the emission of an inelastically scattered photon that may be of higher or lower frequency than the excitation depending on whether the final vibration state of the molecule is higher or lower than the original state. These shifts give information on the vibrational and rotational modes of the sample, which can be related to its material composition. The signal to noise of Raman spectroscopy tends to be weaker compared to other methods such as FTIR.

Hyperspectral imaging creates a spectra for each pixel of an image. Light from an object passes through a dispersing element, such as a prism or a diffraction grating, and then travels to a detector. Optics are typically used in between the dispersing element and the detector to improve image quality and resolution. Hyperspectral imaging may range over a wide range of light wavelengths, including both visible and non-visible light. Multispectral is a subset of hyperspectral imaging that focuses on a few wavelengths of key interest. Hyperspectral imaging is defined by measuring narrow, well defined contiguous wavelengths. Multispectral imaging instead has broad resolution or the wavelengths to be measured are not adjacent to each other. Hyperspectral imaging has been used previously in a wide range of industries. In particular, hyperspectral imaging has been used in aerial mounted surveys to determine mineralogy for oil, gas, and mineral exploration.

FIG. 1 also shows modes of spatial information acquisition, including X-ray CT, NMR, SEM, FIB-SEM, neutron scattering, thin sections and high resolution photography. These can be adapted for use in the present invention from known equipment and manners of use.

The present invention includes the following aspects/embodiments/features in any order and/or in any combination:

1. The present invention relates to a method for determining surface wettability of a sample, comprising:
a) obtaining spectral data on at least one sample;
b) obtaining spatial information on at least one sample;
c) obtaining wettability information on the at least one sample using the spectral data;
d) determining spatially resolved wettability information for the at least one sample using the wettability information and the spatial information, wherein the sample in a) and the sample in b) are the same or are different but have the same or similar composition and structure.
2. The method of any preceding or following embodiment/feature/aspect, wherein the spectral data on the sample is generated by LIBS, TOF-SIMS, SIMS, FTIR, FTIR Microscopy, Raman spectroscopy, Hyperspectral Imaging, or any combinations thereof.
3. The method of any preceding or following embodiment/feature/aspect, wherein the spatial information on the sample is obtained by X-Ray CT scanning, Scanning Electron Microscopy (SEM), Focused Ion Beam-Scanning Electron Microscopy (FIB-SEM), Nuclear Magnetic Resonance (NMR), Neutron Scattering, Thin Sections, High Resolution photography, or any combinations thereof.
4. The method of any preceding or following embodiment/feature/aspect, wherein the sample undergoes spectral measurement and spatial imaging in the same setup, or the sample undergoes spectral measurement and then is transferred to a second setup for spatial imaging, or the sample undergoes spatial imaging and is then transferred to a second equipment for spectral measurement, or the sample undergoes spectral measurement and spatial imaging and one or more intermediate measurements between the two types of measurements. Spectral and spatial measurements may be performed on the exact same samples or two or more samples of similar composition and structure.
5. The method of any preceding or following embodiment/feature/aspect, wherein the wettability information is obtained with determined values for contact angle, surface molecular species, wettability index or indices, or any combinations thereof.
6. The method of any preceding or following embodiment/feature/aspect, comprising estimating the contact angle from spectral measurements on the sample, wherein the contact angle is estimated from molecular species identified from the spectral measurements or wherein univariate or multivariate analysis is used to correlate the spectral measurements to contact angle.
7. The method of any preceding or following embodiment/feature/aspect, comprising determining the surface molecular species wherein molecular species on a surface of the sample identified from spectral measurements are used to correlate the spectral measurements to wettability derived from Amott-Harvey testing, USBM testing, Amott-USBM testing, NMR measurement, or other wettability description metrics, or wherein univariate or multivariate analysis is used to correlate the spectral measurements to molecular species.
8. The method of any preceding or following embodiment/feature/aspect, comprising determining wettability wherein univariate or multivariate analysis is used to correlate the spectral measurements to wettability derived from Amott-Harvey testing, USBM testing, Amott-USBM testing, NMR measurement, or other wettability description metrics.
9. The method of any preceding or following embodiment/feature/aspect, wherein the spatially resolved wettability information is at least one of spatial distribution of wettability indices in 2D or 3D models, spatial distribution of surface molecular species in 2D or 3D models, or spatial distribution of contact angles in 2D or 3D models.
10. The method of any preceding or following embodiment/feature/aspect, wherein the spatial distribution of wettability indices in the 2D or 3D models is determined through image segmentation, assigned manually, determined by capillary pressure simulation or measurements, or determined from previously spatially resolved spectral measurements.
11. The method of any preceding or following embodiment/feature/aspect, wherein the spatial distribution of surface molecular species in the 2D or 3D models is determined through image segmentation, assigned manually, by capillary pressure simulation or measurements, or determined from previously spatially resolved spectral measurements.
12. The method of any preceding or following embodiment/feature/aspect, wherein the spatial distribution of contact angles in the 2D or 3D models is determined through image segmentation, assigned manually, by capillary pressure simulation or measurements, or determined from previously spatially resolved spectral measurements.
13. The method of any preceding or following embodiment/feature/aspect, wherein the sample is a porous sample.
14. The method of any preceding or following embodiment/feature/aspect, wherein the sample is a porous geological sample.
15. A system to perform the method of any preceding claim.
16. A system for determining surface wettability of a sample, comprising i) a spectral data acquisition device for obtaining spectral data on at least one sample; ii) a spatial information acquisition device for obtaining spatial information on at least one sample, wherein the spectral data acquisition device and the spatial information acquisition device are the same device or different devices, and wherein the sample used in i) and the sample used in ii) are the same or are different but have the same or similar composition and structure; iii) one or more computer systems comprising at least one processor and/or computer programs stored on a non-transitory computer-readable medium operable to obtain wettability information on the sample used in i) using the spectral data, and to determine spatially resolved wettability information for the sample or samples used in i) and ii) using the wettability information and the spatial information; and iv) at least one device to display, print, and/or store as a non-transitory storage medium, results of the computations.

The present invention can include any combination of these various features or embodiments above and/or below as set forth in sentences and/or paragraphs. Any combination of disclosed features herein is considered part of the present invention and no limitation is intended with respect to combinable features.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A method for determining surface wettability of a sample, comprising:
a) obtaining spectral data on at least one sample;
b) obtaining spatial information on at least one sample;
c) obtaining wettability information on the at least one sample using the spectral data;
d) determining spatially resolved wettability information for the at least one sample using the wettability information and the spatial information, wherein the sample in a) and the sample in b) are the same or are different but have the same or similar composition and structure.

2. The method of claim 1, wherein the spectral data on the sample is generated by LIBS, TOF-SIMS, SIMS, FTIR, FTIR Microscopy, Raman spectroscopy, Hyperspectral Imaging, or any combinations thereof.

3. The method of claim 1, wherein the spatial information on the sample is obtained by X-Ray CT scanning, Scanning Electron Microscopy (SEM), Focused Ion Beam-Scanning Electron Microscopy (FIB-SEM), Nuclear Magnetic Resonance (NMR), Neutron Scattering, Thin Sections, High Resolution photography, or any combinations thereof.

4. The method of claim 1, wherein the sample undergoes spectral measurement and spatial imaging in the same setup, or the sample undergoes spectral measurement and then is transferred to a second setup for spatial imaging, or the sample undergoes spatial imaging and is then transferred to a second equipment for spectral measurement, or the sample undergoes spectral measurement and spatial imaging and one or more intermediate measurements between the two types of measurements.

5. The method of claim 1, wherein the wettability information is obtained with determined values for contact angle, surface molecular species, wettability index or indices, or any combinations thereof.

6. The method of claim 5, further comprising estimating the contact angle from spectral measurements on the sample, wherein the contact angle is estimated from molecular species identified from the spectral measurements or wherein univariate or multivariate analysis is used to correlate the spectral measurements to contact angle.

7. The method of claim 5, further comprising determining the surface molecular species wherein molecular species on a surface of the sample identified from spectral measurements are used to correlate the spectral measurements to wettability derived from Amott-Harvey testing, USBM testing, Amott-USBM testing, or NMR measurement, or wherein univariate or multivariate analysis is used to correlate the spectral measurements to molecular species.

8. The method of claim 5, further comprising determining wettability wherein univariate or multivariate analysis is used to correlate the spectral measurements to wettability derived from Amott-Harvey testing, USBM testing, Amott-USBM testing, NMR measurement, or other wettability description metrics.

9. The method of claim 1, wherein the spatially resolved wettability information is at least one of spatial distribution of wettability indices in 2D or 3D models, spatial distribution of surface molecular species in 2D or 3D models, or spatial distribution of contact angles in 2D or 3D models.

10. The method of claim 9, wherein the spatial distribution of wettability indices in the 2D or 3D models is determined through image segmentation, assigned manually, determined by capillary pressure simulation or measurements, or determined from previously spatially resolved spectral measurements.

11. The method of claim 9, wherein the spatial distribution of surface molecular species in the 2D or 3D models is determined through image segmentation, assigned manually, by capillary pressure simulation or measurements, or determined from previously spatially resolved spectral measurements.

12. The method of claim 9, wherein the spatial distribution of contact angles in the 2D or 3D models is determined through image segmentation, assigned manually, by capillary pressure simulation or measurements, or determined from previously spatially resolved spectral measurements.

13. The method of claim 1, wherein the sample is a porous sample.

14. The method of claim 1, wherein the sample is a porous geological sample.

15. A system for determining surface wettability of a sample, comprising i) a spectral data acquisition device for obtaining spectral data on at least one sample; ii) a spatial information acquisition device for obtaining spatial information on at least one sample, wherein the spectral data acquisition device and the spatial information acquisition device are the same device or different devices, and wherein the sample used in i) and the sample used in ii) are the same or are different but have the same or similar composition and structure; iii) one or more computer systems comprising at least one processor and/or computer programs stored on a non-transitory computer-readable medium operable to obtain wettability information on the sample used in i) using the spectral data, and to determine spatially resolved wettability information for the sample or samples used in i) and ii) using the wettability information and the spatial information; and iv) at least one device to display, print, and/or store as a non-transitory storage medium, results of the computations.
